(19) United States
(12) Patent Application Publication
(10) Pub. No.: US 2016/0250113 A1
(43) Pub. Date: Sep. 1, 2016

(54) BLONDING COMPOSITION TABLETS

(71) Applicant: Henkel AG & Co. KGaA, Duesseldorf (DE)

(72) Inventors: Udo Erkens, Neuss-Grimlinghausen (DE); Burkhard Mueller, Duesseldorf (DE); Rainer Paulus, Hilden (DE); Thomas Paulus, Duesseldorf (DE)

(73) Assignee: Henkel AG & Co. KGaA, Duesseldorf (DE)

(21) Appl. No.: 15/151,620

(22) Filed: May 11, 2016

Related U.S. Application Data
(63) Continuation of application No. PCT/DE2014/200596, filed on Oct. 28, 2014.

(30) Foreign Application Priority Data
Dec. 6, 2013 (DE) 10 2013 225 169.6

(51) Int. Cl. A61K 8/23; A61K 8/73; A61K 8/891; A61Q 5/08; A61K 8/02

(52) U.S. Cl. CPC A61K 8/23; A61Q 5/08; A61K 8/0216; A61K 8/891; A61K 8/731; A61K 2800/882; A61K 2800/412

(57) ABSTRACT

It has emerged that blonding pastes of relatively high oil content that have been thickened by specific polymers are especially table when the persulfates employed satisfy certain criteria. Blonding tablets including, based on their weight, 10 to 70 wt % of peroxydisulfate(s), 0 to 30 wt % of oil(s) and 1 to 20 wt % of disintegrant(s) made from cellulosic material that before being admixed to components a) and b) and before tableting has been compacted and takes the form within the tablet of compacted granules with a density of 0.3 to 1.5 g/cm³ exhibit increased chemical and physical stability and can be mixed readily with oxidant preparations to produce blonding compositions with stable viscosity.

### FIELD OF THE INVENTION

The present invention generally relates to agents for oxidative color change in the field of cosmetics that are suitable particularly for lightening keratin fibers, in particular human hair.

### BACKGROUND OF THE INVENTION

The oxidizing agents in blonding agents can lighten the hair fibers by oxidatively destroying the pigment melanin in the hair. For a moderate blonding effect, the use of hydrogen peroxide alone as oxidizing agent is sufficient, optionally using ammonia or other alkalizing agents; in order to achieve a stronger blonding effect, a mixture of hydrogen peroxide and peroxydisulfate salts and/or peroxymonosulfate salts are usually used.

For reasons of stability, commercially available blonding agents are usually provided in two separately packaged preparations that are mixed immediately before the application thereof to form a finished application preparation. Conventionally, commercially available blonding agents consist of a liquid oxidizing agent preparation and a powder that includes solid oxidizing agents. These blonding powders have the problem of there being dust during production and when the consumer mixes said powders.

Alternatively, instead of the powder, paste-like agents can be mixed with a liquid oxidizing agent preparation. Paste-like blonding agents mostly include larger amounts of an inert oil, which can lead to stability problems (separation of the solid oxidizing agent from the oil). Even if the peroxydisulfates have not yet been separated off completely, a concentration gradient can occur within the packaging, and therefore different portions from the packaging can cause different lightening after mixing. In addition, the blonding pastes are frequently less effective than the corresponding blonding powders in their performance per unit of weight because of the inert fillers.

In addition, the metering of both powders and pastes by the consumer is often burdensome or difficult. While a person skilled in the art can adapt the lightening result to a specific hair color by varying the amount of powder or paste and oxidizing agent preparation, the consumer that mixes it themselves often finds it difficult to precisely adjust to a desired lightening result when applying it again later.

In order to minimize these problems, a specific amount of blonding agent is desirable. This could be solved by pre-packaged amounts of powder, but this does not solve the problem of dust when mixing. The tablet form of application is considerably more suitable for this purpose and in addition is more widely accepted by consumers.

A tablet has disadvantages as well as advantages: since very stable, i.e. dimensionally stable and fracture-resistant shaped bodies can be produced only by comparatively large pressing power, the shaped body components become highly compressed and the disintegration of the shaped body is delayed as a result.

The problem of the disintegration times of highly compressed shaped bodies being too long is known in particular from pharmaceutics, where certain disintegration auxiliaries, i.e. tablet disintegrants, have been used for a long time to shorten the disintegration times. However, the function of conventional disintegrants over long storage periods cannot be sufficiently ensured for the aggressive ingredients of blonding agents. In addition, the disintegrants have to be problem-free in subsequent mixing in relation to the viscosity, i.e. it is desirable that they not act in a thickening manner.

The problem addressed by the present invention was to further improve the application properties of blonding agents. In this case, the storage stability is intended to be increased in particular, not only the physical stability but also the chemical stability (decomposition of the persalts) being intended to be improved. In this case, tablets are to be provided that disintegrate rapidly, in which the disintegrant does not lose any activity even after a relatively long period of storage and does not hinder the subsequent use e.g. by increasing the viscosity.

It has been shown that tablets made of specific disintegrants and peroxydisulfates and optionally oil(s) solve the above-mentioned problems. An additional advantage is the greater consumer acceptance, since there is no development of dust and reliable portioning is ensured.

Furthermore, other desirable features and characteristics of the present invention will become apparent from the subsequent detailed description of the invention and the appended claims, taken in conjunction with this background of the invention.

### BRIEF SUMMARY OF THE INVENTION

A blonding tablet including, based on its weight, from 10 to 70 wt.% of peroxydisulfate(s), from 0 to 20 wt .% of oil(s), and from 1 to 20 wt. % of disintegrant made of cellulose-containing material which is compacted before being admixed to the components a) and b) and before tableting and is present in the tablet in the form of a compacted granulate having a density of from 0.3 to 1.5 g/cm³.

A method for changing the color of keratin fibers, in which at least two separately packaged preparations (A) and (B), of which preparation (A) includes at least one persulfate and preparation (B) includes at least one oxidizing agent, are combined to form an application mixture, said mixture is applied to the fibers and after a reaction time is rinsed off again, characterized in that preparation (A) is a blonding tablet which, based on its weight, includes from 10 to 70 wt. % of peroxydisulfate(s), from 0 to 20 wt. % of oil(s), and from 1 to 20 wt. % of disintegrant made of cellulose-containing material which is compacted before being admixed to the components a) and b) and before tableting and is present in the tablet in the form of a compacted granulate having a density of from 0.3 to 1.5 g/cm³.

A method for producing blonding tablets in a tablets press, in which a material to be compressed including, based on its weight, from 10 to 70 wt. % of peroxydisulfate(s), from 0 to 20 wt. % of oil(s), and from 1 to 20 wt. % of disintegrant made of cellulose- containing material which is compacted before being admixed to the components a) and b) and is present in the form of a compacted granulate having a density of from 0.3 to 1.5 g/cm³, is filled into a die, the base of which is formed by a lower press die; the material in the die is compressed between the lower press die and an upper press die at a pressing force of from 10 to 60 kN to form a tablet; an additional layer or a plurality of additional layers of material to be compressed are optionally applied to the upper face of the tablet and are compressed to form a (then multi-layer) tablet as in step ii); and the tablet is removed from the die.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

In a first embodiment, the present invention relates to blonding tablets, including, based on their weight,

a) from 10 to 70 wt. % of peroxydisulfate(s),

b) from 0 to 20 wt. % of oil(s);

c) from 1 to 20 wt. % of disintegrant made of cellulose-containing material which is compacted before being admixed to the components a) and b) and before tableting and is present in the tablet in the form of a compacted granulate having a density of from 0.3 to 1.5 g/cm³.

The preparations according to the invention include, as the first essential ingredient, from 10 to 70 wt. % peroxydisulfate(s), preferred blonding tablets including, based on their weight, from 7.5 to 65 wt. %, preferably from 10 to 60 wt. %, more preferably from 20 to 55 wt. %, particularly preferably from 25 to 52.5 wt. %, and in particular from 30 to 40 wt. % of peroxydisulfate(s).

Preferred peroxydisulfates to be used are alkali and ammonium peroxydisulfates, in particular sodium persulfate, potassium persulfate, ammonium persulfate, and mixtures thereof

Preferred blonding tablets include from 5 to 40 wt. %, preferably from 7.5 to 37.5 wt. %, more preferably from 10 to 35 wt. %, particularly preferably from 12.5 to 32.5 wt. %, and in particular from 15 to 30 wt. % of potassium persulfate.

It is most preferred to maintain a greater amount of potassium persulfate than the amount of sodium persulfate and ammonium persulfate that may be used. Furthermore, a specific ratio of potassium persulfate to sodium persulfate has proved to be particularly suitable for obtaining stable and rapidly disintegrating tablets.

In blonding tablets that are preferred according to the invention, the weight ratio of potassium persulfate in the agent to the total amount of peroxydisulfates in the agent is at least 0.2, preferably at least 0.3, more preferably at least 0.4, particularly preferably at least 0.5, and in particular at least 0.6.

The blonding tablets according to the invention can include, as the second ingredient, one or more oils. Said oil(s) is/are preferably liquid under normal conditions.

Cosmetic oils are differentiated into volatile and non-volatile oils. Non-volatile oils are understood to be those oils that have a vapor pressure of less than 2.66 Pa (0.02 mm Hg) at 20° C. and an atmospheric pressure of 1013 hPa. Volatile oils are understood to be those oils that have a vapor pressure of from 2.66 Pa-40000 Pa (0.02 mm-300 mm Hg), preferably from 10-12000 Pa (0.1-90 mm Hg), particularly preferably from 13-3000 Pa, most preferably from 15-500 Pa at 20° C. and an atmospheric pressure of 1013 hPa.

Volatile cosmetic oils are conventionally selected from cyclic silicone oils having the INCI name cyclomethicone. The INCI name cyclomethicone is understood in particular to mean cyclotrisiloxane (hexamethylcyclotrisiloxane), cyclotetrasiloxane (octamethylcyclotetrasiloxane), cyclopentasiloxane (decamethylcyclopentasiloxane), and cyclohexasiloxane (dodecamethylcyclohexasiloxane). These oils have a vapor pressure of approximately 13-15 Pa at 20° C.

A cyclomethicone substitute that is preferred according to the invention is a mixture of C₁₃-C₁₆ isoparaffins, C₁₂-C₁₄ isoparaffins, and C₁₃-C₁₅ alkanes, the viscosity of which is in the range of from 2 to 6 mPas at 25° C. and which has a vapor pressure in the range of from 10 to 150 Pa, preferably from 100 to 150 Pa, at 20° C. Such a mixture is available e.g. under the name SiClone SR-5 from Presperse Inc.

Further preferred volatile silicone oils are selected from volatile linear silicone oils, in particular volatile linear silicone oils having 2-10 siloxane units, such as hexamethyldisiloxane (L₂), octamethyltrisiloxane (L₃), decamethyltetrasiloxane (L₄), as are available for example in the commercial products DC 2-1184, Dow Corning® 200 (0.65 cSt), and Dow Corning® 200 (1.5 cSt) from Dow Corning, and low-molecular-weight phenol trimethicone having a vapor pressure of approximately 2000 Pa at 20° C., as is available from GE Bayer Silicones/Momentive under the name Baysilone Fluid PD 5.

Further products that are preferred according to the invention include at least one volatile non-silicone oil. Preferred volatile non-silicone oils are selected from C₈-C₁₆ isoparaffins, in particular from isononane, isodecane, isoundecane, isododecane, isotridecane, isotetradecane, isopentadecane, and isohexadecane, and mixtures thereof. C₁₀-C₁₃ isoparaffin mixtures are preferred, in particular those having a vapor pressure of from 10-400 Pa, preferably 13-100 Pa, at 20° C.

Furthermore, according to the invention, esters of linear or branched, saturated or unsaturated fatty alcohols having 2-30 carbon atoms with linear or branched, saturated or unsaturated fatty acids having 2-30 carbon atoms that can be hydroxylated are particularly preferable as a cosmetic oil. Esters of linear or branched, saturated fatty alcohols having 2-5 carbon atoms with linear or branched, saturated or unsaturated fatty acids having 10-18 carbon atoms that can be hydroxylated are preferred. Preferred examples therefor are isopropyl palmitate, isopropyl stearate, isopropyl myristate, 2-hexyldecyl stearate, 2-hexyldecyl laurate, isodecyl neopentanoate, isononyl isononanoate, 2-ethylhexyl palmitate, and 2-ethylhexyl stearate. Isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate, ethylene glycol dipalmitate, n-hexyl laurate, n-decyl oleate, oleyl oleate, oleyl erucate, erucyl oleate, C₁₂-C₁₅ alkyl lactate, and di-C₁₂-C₁₃ alkyl malate, and the benzoic acid esters of linear or branched C₈-C₂₂ alkanoles are likewise preferred. Benzoic acid C₁₂-C₁₅ alkyl ester, e.g. available as the commercial product Finsolv® TN (C₁₂-C₁₅ alkyl benzoate), and benzoic acid isostearyl ester, e.g. available as Finsolv® SB, 2-ethylhexyl benzoate, e.g. available as Finsolv® EB, and benzoic acid 2-octyldodecyl ester, e.g. available as Finsolv® BOD, are particularly preferred.

Particularly advantageous is the use of isopropyl esters of C₁₂-C₁₈ carboxylic acids, in particular the use of isopropyl myristate, and particularly preferably mixtures of isopropyl myristate having C₁₀-C₁₃ isoparaffin mixtures, preferably having a vapor pressure of from 10-400 Pa at 20° C.

An additional particularly preferred ester oil is triethyl citrate. Additional products that are preferred according to the invention include triethyl citrate and at least one C₈-C₁₆ isoparaffin, selected from isononane, isodecane, isoundecane, isododecane, isotridecane, isotetradecane, isopentadecane, and isohexadecane, and mixtures of said isoparaffins. Additional products that are preferred according to the invention include triethyl citrate and at least one C₈-C₁₆ isoparaffin, selected from isononane, isodecane, isoundecane, isododecane, isotridecane, and mixtures of said C₈-C₁₆ isoparaffins. Additional products that are preferred according to the invention include triethyl citrate and a mixture of isodecane, isoundecane, isododecane, and isotridecane.

The expression “triglyceride” used below means “glycerol triester”. Additional non-volatile oils that are preferred according to the invention are selected from triglycerides of linear or branched, saturated or unsaturated, optionally hydroxylated C₈-C₃₀ fatty acids, if these are liquid under normal conditions. The use of natural oils, e.g. soya oil, cottonseed oil, sunflower oil, palm oil, palm kernel oil, linseed oil, almond oil, castor oil, corn oil, rapeseed oil, olive oil, sesame oil, safflower oil, wheat germ oil, peach kernel oil, and the liquid proportions of coconut oil and the like can be particularly suitable. Synthetic triglyceride oils, in particular capric/caprylic triglycerides, e.g. the commercial products Myritol® 318 or Myritol® 331 (BASF/Cognis) having unbranched fatty acid esters and glyceryl triisostearin and glyceryl tri(2-ethylhexanoate) having branched fatty acid esters are particularly preferred. Such triglyceride oils preferably make up a proportion of less than 50 wt. % to the total weight of all the cosmetic oils in the product according to the invention.

Additional non-volatile non-silicone oils that are particularly preferred according to the invention are selected from the dicarboxylic acid esters of linear or branched C₂-C₁₃ alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl) succinate.

Additional non-volatile non-silicone oils that are particularly preferred according to the invention are selected from the symmetrical, asymmetrical or cyclic esters of carbonic acids having C₆-C₂₀ alcohols, e.g. di-n-caprylyl carbonate (Cetiol® CC) or di-(2-ethylhexyl) carbonate (Tegosoft DEC). Esters of carboxylic acids having C₁-C₅ alcohols, e.g. glycerine carbonate or propylene carbonate, are compounds that are not suitable as a cosmetic oil, however.

Additional oils that may be preferred according to the invention are selected from the esters of dimers of unsaturated C₁₂-C₂₂ fatty acids (dimer fatty acids) having monovalent linear, branched, or cyclic C₂-C₁₈ alkanols or having polyvalent linear or branched C₂-C₀₅ alkanols. The total weight of dimer fatty acid esters is particularly preferably from 0.5-10 wt. %, preferably 1-5 wt. %, in each case based on the weight of the total water-in-oil emulsion, without taking into account the weight of the propellant.

Additional cosmetic oils that are particularly preferred according to the invention are selected from non-volatile silicone oils. Non-volatile silicone oils that are preferred according to the invention are selected from linear polyalkyl siloxanes having a kinematic viscosity of at least 5 cSt to 2000 cSt at 25° C., in particular selected from linear polydimethyl siloxanes having a kinematic viscosity of from 5 cSt to 2000 cSt, preferably from 10 to 350 cSt, particularly preferably 50-100 cSt, at 25° C., as available e.g. under the trade names Dow Corning® 200 or Xiameter PMX from Dow Corning or Xiameter, respectively. Additional preferred non-volatile silicone oils are phenyl trimethicones having a kinematic viscosity of from 10 to 100 cSt, preferably from 15-30 cSt, at 25° C., and cetyl dimethicones.

Agents that are preferred according to the invention include at least one non-volatile silicone oil that is preferably selected from linear polyalkyl siloxanes having a kinematic viscosity of from 5 cSt-2000 cSt, preferably from 10-350 cSt, particularly preferably 50-100 cSt, at 25° C., in particular selected from linear polydimethyl siloxanes having a kinematic viscosity of from 5 cSt-2000 cSt, preferably 10-350 cSt, particularly preferably 50-100 cSt, at 25° C. in a total amount of from 0.1-30 wt. %, preferably 1-24 wt. %, particularly preferably 2-18 wt. %, most preferably 4-10 wt. %, in each case based on the weight of the total agent.

Paraffin oils are likewise suitable as an oil in the tablets according to the invention. The paraffin oils are understood to be mixtures of saturated, aliphatic hydrocarbons that are liquid at room temperature. Agents that are preferred according to the invention therefore include at least one paraffin oil.

Of the oils mentioned above, some have proved to be particularly suitable since they guarantee the physical and chemical stability of the blonding tablets over long periods of time and are especially compatible with the additional ingredients according to the invention. Blonding tablets that are preferred according to the invention are characterized in that they include from 2.5 to 17.5 wt. %, preferably from 3.5 to 15 wt. %, more preferably from 5 to 12.5 wt. %, particularly preferably from 6 to 11 wt. %, and in particular from 7.5 to 10 wt. %, of oil(s) from the group containing paraffin oil, polyisobutene, alkyl benzoates, isopropyl palmitate, isohexadecane, isododecane, isononyl isononanoate.

Further preferred blonding tablets according to the invention include from 2.5 to 17.5 wt. %, preferably from 3.5 to 15 wt. %, more preferably from 5 to 12.5 wt. %, particularly preferably from 6 to 11 wt. %, and in particular from 7.5 to 10 wt. %, of paraffin oil.

As the third essential ingredient, the blonding tablets according to the invention include from 1 to 20 wt. % of disintegrant made of cellulose-containing material which is compacted before being admixed to the components a) and b) and before tableting and is present in the tablet in the form of a compacted granulate having a density of from 0.3 to 1.5 g/cm3.

The cellulose-containing material is in this case compacted before being admixed to the additional ingredients, i.e. the ingredients a) and b). In this case, the expression “compacting” is intended to mean the exertion of a pressure on the cellulose-containing material, which reduces the volume of the cellulose-containing material without destroying the fibers. The particles are therefore deformed during compacting, in contrast to aggregation, in which the particles are merely agglomerated without substantially changing their shape. The ingredients are to be compacted in this sense before admixing thereto the disintegrant thus produced. If the pellet comes into contact with water or any other liquid, the cellulose-containing material springs from its compact state back into a state having an open and unstressed volume.

“Compacting” within the meaning of the present invention can be granulation or extrusion; the compacting is preferably roll compacting, in which the cellulose-containing material is fed to two counter-rotating rollers having parallel axes of rotation and is compressed when passing between the rollers.

During compacting, granules are produced from the starting material during or after the compacting, which granules form larger aggregates of a plurality of starting particles. These larger aggregates, i.e. the granules, are admixed to the ingredients and the mixture is compressed to form the pellets.

After the compacting process, the compacted granulate to be used is intended to have a density of from 0.3 to 1.5 g/cm³. On the one hand, a sufficient disintegrating effect is achieved in this way, and on the other hand this range ensures that a chemical deactivation of the disintegrant is stopped by the aggressive tablet components. Moreover, the oils that are conventionally incompatible with disintegrants can also be in the tablets without loss of physical efficiency, this not being the case with conventional disintegrants.

The “cellulose-containing materials” to be used as the disintegrant according to the invention are preferably those in which the cellulose is still present in an at least predominantly chemically unchanged form. A particle size of the starting material which is present in larger granules after compacting of from 40-60 μm is expedient. Extremely fine cellulose-containing starting materials of this grain fineness can be produced with acceptable comminution effort and in practice do not lead to problems in terms of viscosity or transparency of the application mixture when it is used later.

The compacted particles of the cellulose-containing material can have a particle size of from 0.2 to 6.0 mm, in particular from 0.3 to 1.5 mm, the most expedient particle size also depending on the size of the pellet.

The dispersion properties of the cellulose-containing material can be enhanced if said material is fibrillated at least in part, i.e. is comminuted except for bundles that are each made up of a few parallel cellulose fibers.

During the development work, two types of cellulose-containing material proved to be excellent, specifically TMP (thermomechanical pulp) and CTMP (chemi-thermomechanical pulp). These are two types of mechanical pulp. In the TMP method, wood chips are fibrillated under vapor pressure at approximately 130° C. in pressurized refiners to form TMP. CTMP is produced when chemicals are used in wood chip pre-vaporization. Although in the TMP and CTMP mechanical pulps a certain amount of leaching of the material has taken place, the lignins, resins and other accompanying substances in the wood are not completely removed, in particular not as completely as in the production of cellulose. These mechanical pulps are therefore cellulose-containing materials that still have some of the properties of wood.

Blonding tablets that are particularly preferred according to the invention are characterized in that they include from 2.5 to 20 wt. %, preferably from 3.5 to 18 wt. %, more preferably from 5 to 16 wt. %, particularly preferably from 7 to 15 wt. %, and in particular from 9 to 12 wt. %, of disintegrant made of cellulose-containing material, in which more than 70 wt. %, preferably more than 80 wt. %, more preferably more than 90 wt. %, and in particular more than 99 wt. %, of the compacted granulate of the cellulose-containing material has a particle size of from 0.2 to 6.0 mm, preferably from 0.4 to 1.5

MM.

Blonding tablets that are further preferred according to the invention are characterized in that they include from 2.5 to 20 wt. %, preferably from 3.5 to 18 wt. %, more preferably from 5 to 16 wt. %, particularly preferably from 7 to 15 wt. %, and in particular from 9 to 12 wt. %, of disintegrant made of cellulose-containing material, in which the particle size of the cellulose-containing starting material is from 20 to 200 preferably from 40 μm to 60 μm.

The blonding agent tablets according to the invention can include alkalizing agents. Preferred alkalizing agents are for example ammonia, alkanolamines, basic amino acids, and inorganic alkalizing agents such as alkali (earth) metal hydroxides, alkali (earth) metal metasilicates, alkali (earth) metal silicates, alkali (earth) metal phosphates, alkali (earth) metal metaphosphates, and alkali (earth) metal hydrogen phosphates. The metal ions used are preferably lithium, sodium, and/or potassium.

Inorganic alkalizing agents that can be used according to the invention are preferably selected from calcium hydroxide, barium hydroxide, sodium phosphate, sodium hexametaphosphate, potassium phosphate, sodium silicate, potassium silicate, sodium metasilicate, potassium metasilicate, magnesium silicate, sodium carbonate, and potassium carbonate. More preferred are sodium silicate, sodium metasilicate, and/or sodium hexametaphosphate.

Alkalizing agents that can be used according to the invention are preferably selected from alkanolamines from primary, secondary, or tertiary amines having a C₂-C₆ alkyl basic structure, bearing at least one hydroxyl group. Particularly preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol (monoisopropanolamine), 1-aminobutan-2-ol, 1-aminopentan-2-ol, 1-aminopentan-3-ol, 1-aminopentan-4-ol, 2-amino-2-methylpropanol, 2-amino-2-methylbutanol, 3-amino-2-methylpropan-1-ol, 1-amino-2-methylpropan-2-ol, 3-aminopropan-1,2-diol, 2-amino-2-methylpropan-1,3-diol, 2-amino-2-ethyl-1,3-propanediol, N,N-dimethylethanolamine, methylglucamine, triethanolamine, diethanolamine, and triisopropanolamine.

The basic amino acids that can be used as an alkalizing agent according to the invention are preferably selected from the group formed by L-arginine, D-arginine, D/L-arginine, L-lysine, D-lysine, D/L-lysine, L-ornithine, D-ornithine, D/L-ornithine, L-histidine, D-histidine, and/or D/L-histidine. L-arginine, D-arginine, and/or D/L-arginine are particularly preferably used as an alkalizing agent within the meaning of the invention.

If the ready-to-use mixtures include alkalizing agents, preparations that include alkalizing agent in an amount of from 1 to 70 wt. %, in particular of from 10 to 40 wt. %, based in each case on the total weight of the ready-to-use agent, are preferred according to the invention.

The preparations according to the invention may additionally include at least one additional bleach enhancer, which is different from the inorganic persalts.

Compounds which produce aliphatic peroxycarboxylic acids preferably having from 1 to 10 C atoms, in particular from 2 to 4 C atoms, and/or optionally substituted perbenzoic acid under perhydrolysis conditions may be used as bleach enhancers. Substances bearing O- and/or N-acyl groups of the aforementioned number of C atoms and/or optionally substituted benzoyl groups are suitable. Polyacylated alkylendiamines, in particular tetraacetylethylenediamine (TAED), acylated triazine derivates, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenol sulfonates, in particular n-nonanoyl or isononanoyl oxybenzene sulfonate (n- or iso-NOBS), carboxylic acid anhydrides, in particular phthalic acid anhydride, acylated polyhydric alcohols, in particular triacetin, ethylene glycol diacetate, and 2,5-diacetoxy-2,5-dihydrofuran are preferred.

The invention secondly relates to a method for changing the color of keratin fibers, in which at least two separately packaged preparations (A) and (B), of which preparation (A) includes at least one persulfate and preparation (B) includes at least one oxidizing agent, are combined to form an application mixture, said mixture is applied to the fibers and after a reaction time is rinsed off again, wherein preparation (A) is a blonding tablet which, based on its weight, includes

a) from 10 to 70 wt. % of peroxydisulfate(s),

b) from 0 to 20 wt. % of oil(s);

c) from 1 to 20 wt. % of disintegrant made of cellulose-containing material which is compacted before being admixed to the components a) and b) and before tableting and is present in the tablet in the form of a compacted granulate having a density of from 0.3 to 1.5 g/cm³.

The ready-to-use agents are produced immediately before being applied to the hair by mixing the two preparations (A) and (B) and optionally a third preparation (C) and/or additional preparations. In ready-to-use agents that are mixed from more than two preparations to form a finished application mixture, it may be irrelevant whether two preparations are first mixed together and then the third preparation is added and mixed in, or whether all the preparations are combined at the same time and then mixed together. Mixing can take place by stirring in a dish or a beaker or by agitation in a closable receptacle.

The term “immediately” is in this case understood to mean the time period of a few seconds to an hour, preferably up to 30 min, in particular up to 15 min.

The agents according to the invention are used in a method for lightening keratin fibers, in particular human hair, in which the agent is applied to the keratin-containing fibers, left on the fibers at a temperature of from room temperature to 45° C. for a reaction time of from 10 to 60 minutes and then rinsed off again with water or washed out with a shampoo.

The reaction time of the ready-to-use lightening agents is preferably from 10 to 60 min, in particular from 15 to 50 min, particularly preferably from 20 to 45 min. During the reaction time of the agent on the fibers, it may be advantageous to assist the lightening process by introducing heat. Heat can be introduced by means of an external heat source, such as using a hot-air blower and also, in particular when lightening the hair on living test subjects, by means of the body temperature of the test subject. In the latter option, the parts to be lightened are conventionally covered with a hood-type hair drier. A reaction phase at room temperature is likewise claimed. Preferably, the temperature during the reaction time is between 20° C. and 40° C., in particular between 25° C. and 38° C. The lightening agents produce good blonding and lightening results even at physiologically acceptable temperatures of less than 45° C.

At the end of the reaction time, the remaining lightening preparation is rinsed from the hair using water or a cleaning agent. As a cleaning agent, commercially available shampoo can be used in particular, it in particular being possible to omit the cleaning agent and to carry out the rinsing process using tap water if the lightening agent has a carrier including a high level of surfactant.

The preferred embodiments of the first subject matter of the invention also apply mutatis mutandis to the second subject matter of the invention.

The invention thirdly relates to a method for producing blonding tablets in a tablets press, in which

i) a material to be compressed including, based on its weight,
a) from 10 to 70 wt. % of peroxydisulfate(s),
b) from 0 to 20 wt. % of oil(s);
c) from 1 to 20 wt. % of disintegrant made of cellulose-containing material which is compacted before being admixed to the components a) and b) and is present in the form of a compacted granulate having a density of from 0.3 to 1.5 g/cm³,
is filled into a die, the base of which is formed by a lower press die;

ii) the material in the die is compressed between the lower press die and an upper press die at a pressing force of from 10 to 60 kN to form a tablet;

iii) an additional layer or a plurality of additional layers of material to be compressed are optionally applied to the upper face of the tablet and are compressed to form a (then multi-layer) tablet as in step ii) and
iv) the tablet is removed from the die.

The material to be compressed in step i) corresponds to the later composition of the blonding agent tablets according to the invention. The pressing pressure in step ii) is produced by said pressing force per pressing surface area, i.e. is dependent on the surface area of the shaped body to be compressed.

Tablet pressing surface areas in the range of from 5 to 40 cm² have proved to be particularly suitable, surface areas of from 6 to 35 cm², preferably from 7 to 30 cm², more preferably from 8 to 25 cm², and in particular from 9 to 15 cm², being most preferred.

Particularly preferred pressing pressures are therefore in the range of from 1.2 to 4.5 kN cm⁻².

The preferred embodiments of the first subject matter of the invention also apply mutatis mutandis to the third subject matter of the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A blonding tablet including, based on its weight,
a) from 10 to 70 wt. % of peroxydisulfate(s),
b) from 0 to 20 wt. % of oil(s),
c) from 1 to 20 wt. % of disintegrant made of cellulose-containing material which is compacted before being admixed to the components a) and b) and before tableting and is present in the tablet in the form of a compacted granulate having a density of from 0.3 to 1.5 g/cm³.

2. The blonding tablet according to claim 1, wherein the peroxydisulfate(s) comprises from 7.5 to 65 wt. % of the blonding tablet.

3. The blonding tablet according to claim 1, wherein the peroxydisulfate(s) includes potassium persulfate.

4. The blonding tablet according to claim 3, wherein the potassium persulfate comprises 5 to 40 wt % of the blonding tablet.

5. The blonding tablet according to claim 3, wherein the weight ratio of potassium persulfate to the total amount of peroxydisulfates in the blonding tablet is at least 0.2.

6. The blonding tablet according to claim 3, wherein the weight ratio of potassium persulfate to the total amount of peroxiydisulfates in the blonding tablet is at least 0.4.

7. The blonding tablet according to claim 1, wherein the oils comprise 2.5 to 17.5 wt. % of the blonding tablet.

8. The blonding tablet according to claim 1, wherein the oil includes at least one oil selected from the group consisting of paraffin oil, polyisobutene, alkyl benzoates, isopropyl palmitate, isohexadecane, isododecane, and isononyl isononanoate.

9. The blonding tablet according to claim 1, wherein the oil is paraffin oil.

10. The blonding tablet according to claims 1, further comprising 0.1-30 wt. % of non-volatile silicone oil(s).

11. The blonding tablet according to claim 10, wherein the non-volatile silicone oil(s) includes a linear polyalkylsiloxane having a kinematic viscosity of 5-2000 cSt. at 25° C.

12. The blonding tablet according to claim 1, wherein the compacted granulate of the cellulose-containing material comprises 2.5 to 17.5 wt. % of the blonding tablet.

13. The blonding tablet according to claim 1, wherein the compacted granulate of the cellulose-containing material has a particle size of from 0.2 to 6.0 mm.

14. The blonding tablet according to claim 1, characterized in that it includes from 2.5 to 17.5 wt. %, of disintegrant made of cellulose-containing material, in which the particle size of the cellulose-containing starting material is from 20 to 200 μm.

15. The blonding tablet according to claim 1, characterized in that the compacted particles of the cellulose-containing material have a particle size of from 0.2 to 6.0 mm.

16. A method for changing the color of keratin fibers, in which at least two separately packaged preparations (A) and (B), of which preparation (A) includes at least one persulfate and preparation (B) includes at least one oxidizing agent, are combined to form an application mixture, said mixture is applied to the fibers and after a reaction time is rinsed off again, characterized in that preparation (A) is a blonding tablet which, based on its weight, includes
a) from 10 to 70 wt. % of peroxydisulfate(s),
b) of from 0 to 20 wt. % oil(s),
c) from 1 to 20 wt. % of disintegrant made of cellulose-containing material which is compacted before being admixed to the components a) and b) and before tableting and is present in the tablet in the form of a compacted granulate having a density of from 0.3 to 1.5 g/cm³.

17. The method according to claim 16, characterized in that the reaction time is from 10 to 60 min.

18. The method according to claim 16, characterized in that the temperature during the reaction time is between 20° C. and 40° C.

19. A method for producing blonding tablets in a tablets press, in which
i) a material to be compressed including, based on its weight,
a) from 10 to 70 wt. % of peroxydisulfate(s),
b) from 0 to 20 wt. % of oil(s),
c) from 1 to 20 wt. % of disintegrant made of cellulose-containing material which is compacted before being admixed to the components a) and b) and is present in the form of a compacted granulate having a density of from 0.3 to 1.5 g/cm³,
is filled into a die, the base of which is formed by a lower press die;
ii) the material in the die is compressed between the lower press die and an upper press die at a pressing force of from 10 to 60 kN to form a tablet;
iii) an additional layer or a plurality of additional layers of material to be compressed are optionally applied to the upper face of the tablet and are compressed to form a (then multi-layer) tablet as in step ii); and
iv) the tablet is removed from the die.

20. The method according to claim 19, characterized in that the tablet pressing surface area is in the range of from 5 to 40 cm².
